(19) United States
(12) Patent Application Publication
(10) Pub. No.: US 2010/0268126 A1
(43) Pub. Date: Oct. 21, 2010

(54) DEVICE TO BE WORN BY A PATIENT FOR CONTROLLING MOVEMENTS OF THE PATIENT

(76) Inventor: Hans-Peter KLOSE, Stuttgart (DE)

Correspondence Address: KENYON & KENYON LLP, ONE BROADWAY, NEW YORK, NY, 10004, US

(21) Appl. No.: 12/763,730

(22) Filed: Apr. 20, 2010

(30) Foreign Application Priority Data
Apr. 21, 2009 (DE) 10 2009 002 547.2

(51) Int. Cl. A61B 5/103

(52) U.S. Cl. 600/595

(57) ABSTRACT

A device to be worn by a patient for controlling movements of the patient is described. As to the device to be worn by a patient for controlling movements of the patient, the device has a sensor unit for recording measured values regarding a movement of the patient, an evaluation unit for determining a measure characterizing or describing the movement, a comparator for comparing the determined measure with a specifiable comparison measure and for determining a deviation, as well as a signal unit for displaying a determined deviation.

### RELATED APPLICATION INFORMATION

The present application claims priority to and the benefit of German patent application no. 10 2009 002 547.2, which was filed in Germany on Apr. 21, 2009, the disclosure of which is incorporated herein by reference.

### FIELD OF THE INVENTION

The present invention relates to a device to be worn by a patient for controlling movements of the patient.

BACKGROUND INFORMATION

In the rehabilitation field, the correct execution of movements is essential for the long-term success of therapeutic measures, such as physical therapy following an operative hip- or knee-joint intervention. In the area of neurological diseases as well, diseases are known such as a stroke or damage to the inner ear caused by otitis media, for example, which lead to a loss of the sense of balance. It may be relearned or improved also by suitable training together with movement or position feedback to the patient.

In the prevention field, especially in the case of older people, the restoration of correct movements by physical therapy is frequently an effective measure, in particular for preventing falls.

All of these cases require intensive and long-term treatment under the supervision of a physical therapist, for example, which can be provided only in individual cases, however.

Especially in view of the demographic development, it must be assumed that the prevalence or incidence of the described cases will increase.

Therefore, it is desirable to have an option for monitoring movements of a patient, which can be provided independently of a physical therapy session or independently of treatment, in particular.

### SUMMARY OF THE INVENTION

This objective may be achieved by a device having the features described herein. Advantageous developments are also described in the following description.

A device according to the exemplary embodiments and/or exemplary methods of the present invention to be worn by a patient includes a sensor unit for recording measured values pertaining to an in particular specifiable or selectable movement of the patient, an evaluation unit for determining a measure characterizing or describing the movement, a comparator for comparing the determined measure with a specifiable comparison measure and for determining a deviation, and also a signal unit for indicating a determined deviation. The measure characterizing the movement may be a value such as an average value, a median, a derived value, such as the gradient of a time characteristic, but also value sequences, a time characteristic, a pattern etc. A comparison may take the form of a threshold comparison, pattern detection etc.

Using the approach according to the exemplary embodiments and/or exemplary methods of the present invention makes it possible to control the movements of the patients, in particular in a continuous manner and independently of therapy or treatment sessions. The patient carries the device with him, which monitors a selected movement of the patient in order to check whether it conforms to a specified comparison measure. If this is not the case, then this fact will be signaled to the patient optically, acoustically or haptically, for instance. Such technical support, in particular, eases the workload of therapists, since it allows the patients to practice on their own without being continuously monitored by the therapist, or it allows monitoring of the patients. More specifically, a therapist is able to tend to more patients, which, in view of the expected rate of increase (e.g., of 50 to 70% for strokes by the year 2050) and simultaneously less available skilled personnel, has a cost-reducing effect and maintains the level of medical care. Supply problems, especially in rural areas, are able to be solved more optimally. In particular, the therapist is able to receive quantitative data regarding the rehabilitation success, which allows an optimization of the individual rehabilitation program. The patient receives qualitative and/or quantitative feedback regarding her movements, which makes it possible for the patient to check them herself and to improve them.

A use of the device according to the present invention takes place in the rehabilitation following operative hip or knee joint replacement. After a hip operation, for example, a patient is instructed to put only limited pressure on the operated leg for approximately two to four weeks. Toward this end, he is intentionally taught an “asymmetrical” way of walking, which is characterized by an asymmetrical time behavior of the left-right step timing relative to the right-left step timing. This time behavior is able to be specified to the device as a comparison measure. The device records the particular step times and compares them to the specified comparison measure, in particular in the form of a pattern comparison. If the asymmetry of the step timing does not lie within the framework specified by a physical therapist, for example, then this will be signaled to the patient. As a result, the patient is able to make more of an effort at adopting the asymmetrical gait. In the further course of rehabilitation, increasingly more weight may be placed on the operated leg, which is to produce a more symmetrical gait. At this point, however, the patient typically has already internalized the asymmetrical gait and therefore no longer perceives it as such. The device now includes the patterns of a symmetrical gait as comparison measure and is able to alert the patient to the occurrence of an asymmetrical walking pattern.

Another advantageous possibility for utilizing the exemplary embodiments and/or exemplary methods of the present invention is movement training for the prevention of falls. It is known that the correct movement of the feet when walking diminishes, especially in older people. One such manifestation is a shambling gait, for instance, in which the feet are no longer lifted off the floor to any significant extent. This increases the danger of falls, for example, since even minimal thresholds in the house or in the environment turn into trip hazards. A sensor, fixed in place on the foot or on the lower leg, detects the occurrence of such shambling, and the device subsequently signals to the wearer that the feet have to be picked up more. The attention is focused on the correct execution (e.g., accompanied by the physical therapist), and the correct movement is relearned, which reduces the risk of falls and injuries.

In the same way the exemplary embodiments and/or exemplary methods of the present invention may advantageously be used in reestablishing the sense of balance. A person's sense of balance may be lost as a result of various diseases. This makes it impossible for the patient to stabilize the body in an erect posture, such as standing without support; instead, the patient falls after a short period of time. Various research (e.g., according to Bach-y-Rita) indicate that it is possible to re-teach the sense of balance to a patient with technical support. For this purpose the device signals to the patient his relative movement with respect to the perpendicular and conveys the type of deviation (e.g., tilting to the left rear) by corresponding feedback. The sensor unit is affixed on the head of the patient in a marked position in order to record this deviation.

The device advantageously includes a storage unit for storing the measured values and/or the determined measures. This storage unit may be used particularly for long-term recording, so that a therapist, for example, is able to judge the course of rehabilitation or the quality of the patients' movements or their progress at regular or irregular intervals.

The device may be equipped with a transmission unit for the wireless transmission of the measured values and/or the determined measures to a remote unit. In particular, the transmission unit enables a tele-medical use of the device.

For example, alarm-triggering parameters may be transmitted to a tele-medical center or to an appropriately equipped physical therapist for objectification of the rehabilitation progress and for optimization of the rehabilitation program.

The sensor unit may have at least one acceleration sensor, in particular a triaxial acceleration sensor. In addition, an expansion by a gyroscope is contemplated. When using inertial sensors, it is useful to fix them in place on the patient at defined locations. The movements of the patients are able to be monitored in a simple and reliable manner by providing corresponding sensors.

The signal unit is advantageously designed as visual signal unit, e.g., as indicator, display, light signal transmitter, LED etc. In order to inform the patient of a deviating movement in a timely manner, the visual signal unit should be situated in the visual field of the patient. Small light signal transmitters such as light diodes, for example, which are affixed on spectacle frames, for instance, and emit light signals when detecting a movement deviation, lend themselves to this purpose.

An acoustic signal unit may advantageously be provided as well, which is directly integrated into the sensor unit, for example. However, since an acoustic signal output is undesired in certain situations, it is also possible to provide a signal unit that is separate from the sensor unit, which may be provided in the form of a hearing aid or installed in the side pieces of eyeglasses, for instance.

In the same advantageous manner, a haptic signal unit developed as vibrator is provided. It may likewise be integrated in the sensor unit or developed as a unit separate therefrom, for instance in the form of a wristband or a type of plaster, for example.

It is useful if at least two of the mentioned units have an integrated design in order to save transmission means between the individual units. This is to be interpreted such, in particular, that the entire device is implemented in one piece, but also such that individual units of the device have an integrated design, for example in a microprocessor. As a result, a device according to the present invention is robust and easy to place, especially by older people.

However, it is equally useful if at least the signal unit is separate from the sensor unit. This suggests itself especially in the case of a visual signal unit, but for others as well.

Further advantages and refinements of the exemplary embodiments and/or exemplary methods of the present invention are derived from the description and the accompanying drawing.

It is understood that the features mentioned above and the features yet to be described below may be used not only in the combination given in each case but also in other combinations or individually, without departing from the scope of the exemplary embodiments and/or exemplary methods of the present invention.

The exemplary embodiments and/or exemplary methods of the present invention is represented schematically in the drawing in light of an exemplary embodiment, and is described in detail below with reference to the drawing.

### BRIEF DESCRIPTION OF THE DRAWING

FIG. 1 schematically illustrates an exemplary embodiment of a device according to the present invention for controlling movements of a patient.

### DETAILED DESCRIPTION

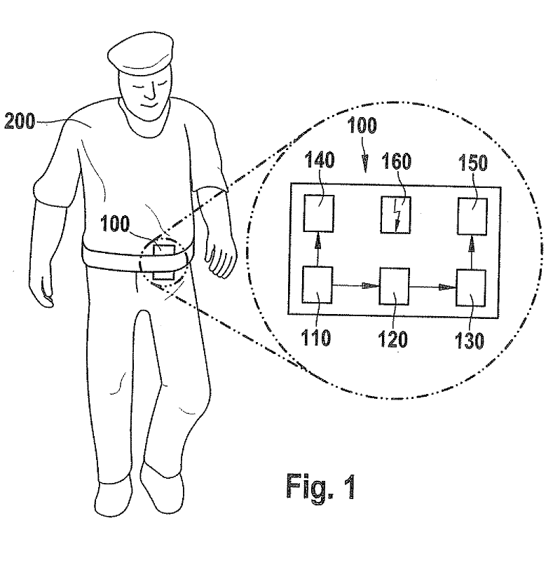

FIG. 1 schematically illustrates an exemplary embodiment of a device according to the present invention for controlling movements of a patient, the device being denoted by 100 as a whole. A patient 200 wears device 100 at the hip and in the illustrated use uses it for controlling the walking movement following a hip joint operation. As described earlier already, in this type of use device 100 monitors the step timing of patient 200 for symmetry or asymmetry.

Device 100 is equipped with a sensor unit for recording measured values regarding the gait of the patient, the sensor unit being implemented as triaxial acceleration sensor 100 in the case at hand. In the development shown, device 100 has an integrated sensor unit 110. In the same way, however, each leg of the patient, for example, may be provided with a separate sensor unit, which is connected to an evaluation unit of the device in a wireless or wirebound manner.

Furthermore, device 100 includes an evaluation unit 120, to which the measured values recorded by sensor unit 110 are transmitted. The evaluation unit is designed to transmit a measure characterizing the movement on the basis of the recorded measured values, the step timing suggesting itself for this purpose.

In addition, device 100 has a comparator 130 for comparing the determined measure with a specified comparison measure. The comparison measure is advantageously specified by a therapist and stored in device 100. The storage may take place in an internal storage unit of comparator 130 or in a separate storage unit. In the illustration shown, for example, a storage unit 140 is provided for storing the measured values, in particular for a long-term analysis, in which the comparison measure may be stored as well.

On the basis of the comparison, comparator 130 determines whether a deviation has occurred between the measure characterizing the movement and the specified comparison measure. This determination is able to be made with the aid of, for instance, a threshold comparison, a pattern comparison, etc. If a deviation and thus an undesired movement of the patient is determined in comparator 130, then a signal unit 150 will be activated in order to indicate this fact to patient 200. Here, signal unit 150 is implemented as vibrator, which signals the deviation to the patient with the aid of a vibrating alarm.

Furthermore, device 100 has an energy source for the supply of device 100, this source being implemented as storage battery 160.

For instance, evaluation unit 120 as well as comparator 130 are able to be integrated in a shared unit such as a microprocessor.

Optionally, device 100 may include a transmission unit (not shown here) for transmitting the measured values and/or the determined measure to a remote unit in a wireless manner, and also for receiving from a remote unit programming data, for example, regarding the specified comparison measure.

It is understood that the figures shown illustrate merely exemplary specific embodiments of the present invention. In addition, any other specific embodiment is conceivable without departing from the framework of this invention.

What is claimed is:

1. A device to be worn by a patient for controlling movements of the patient, comprising:
a sensor unit to record measured values regarding a movement of the patient;
an evaluation unit to determine a measure characterizing or describing the movement;
a comparator to compare the determined measure with a specifiable comparison measure and to determine a deviation; and
a signal unit to display the determined deviation.

2. The device of claim 1, further comprising:
a storage unit to store at least one of the measured values and the determined measures.

3. The device of claim 1, further comprising:
a transmission unit to wirelessly transmit at least one of the measured values and the determined measures to a remote unit.

4. The device of claim 1, wherein the sensor unit includes at least one acceleration sensor.

5. The device of claim 1, wherein the sensor unit includes at least one gyroscope.

6. The device of claim 1, wherein the signal unit includes at least one of a display, a vibrator, a sound signal transmitter and a light signal transmitter.

7. The device of claim 1, wherein at least two of a display, a vibrator, a sound signal transmitter and a light signal transmitter have an integrated configuration.

8. The device of claim 1, wherein at least the signal unit is implemented separately from the sensor unit.

9. The device of claim 1, wherein the sensor unit includes at least one triaxial acceleration sensor.
